(19) United States
(12) Patent Application Publication
(10) Pub. No.: US 2002/0165161 A1
(43) Pub. Date: Nov. 7, 2002

(54) Chemokine-binding proteins for treating congestive heart failure

(75) Inventor: Anthony Allison, Belmont, CA (US)

Correspondence Address: SWANSON & BRATSCHUN L.L.C., 1745 SHEA CENTER DRIVE, SUITE 330, HIGHLANDS RANCH, CO, 80129, US

(73) Assignee: SURROMED, INC., Mountain View, CA

(21) Appl. No.: 10/138,194

(22) Filed: May 2, 2002

Related U.S. Application Data
(60) Provisional application No. 60/288,659, filed on May 4, 2001.
(60) Provisional application No. 60/337,107, filed on Dec. 6, 2001.

(51) Int. Cl. A61K 48/00; A61K 38/17

(52) U.S. Cl. 514/12; 514/44; 424/93.2

(57) ABSTRACT

Chemokine-binding proteins are administered as a treatment for congestive heart failure and related disorders. Evidence indicates that CC chemokines are a factor in the disease pathogenesis; by binding to receptors on heart muscle cells, the chemokines may, through a G-protein-coupled transduction system, increase the concentration of free calcium ions in the cytoplasm. This disrupts the normal calcium cycle required for muscle contraction and relaxation, and is a likely mechanism for development of congestive heart failure. Suitable proteins include poxyirus-encoded CC chemokine inhibitors and promiscuous chemokine receptors; these proteins bind to a number of different CC chemokines with high affinity but generally do not bind to other subfamilies of chemokines.

### CROSS-REFERENCE TO RELATED APPLICATIONS

This application claims the benefit of U.S. Provisional Application No. 60/288,659, “Methods and Materials for Treatment of Congestive Heart Failure and Arrhythmia,” filed May 4, 2001, and U.S. Provisional Application No. 60/337,107, “Methods and Materials for Treating Congestive Heart Failure,” filed Dec. 6, 2001, both of which are hereby incorporated herein by reference.

### FIELD OF THE INVENTION

The present invention relates generally to compositions and methods for treating congestive heart failure and related conditions. More particularly, the invention relates to a protein capable of binding to several CC chemokines contributing to calcium overload of cardiac muscle cells, a factor in the pathogenesis of congestive heart failure.

### BACKGROUND OF THE INVENTION

Congestive heart failure (CHF) is a degenerative condition that occurs when the heart is unable to pump enough blood to meet the needs of the body's tissues. When the left side of the heart fails, fluid backs up (congests) in the lungs and does not reach the necessary tissues. When the right side fails, a much less common condition, fluid entering the heart backs up, causing the veins in the body and surrounding tissue to swell. Congestive heart failure is a common complication of many types of heart disease; in the United States, 400,000 new cases of CHF are diagnosed each year, and 250,00 people die of it each year.

Conventional drug treatment for CHF involves four classes of medications: vasodilators, particularly inhibitors of angiotension-converting enzyme (ACE); inotropics, which increase the heart's ability to contract but have not been shown to prolong life; diuretics, which reduce fluid retention; and beta blockers, which prevent binding of adrenaline to heart cells. All current treatments have significant side effects and do not work well in all patients. Because the pathogenesis of CHF is poorly understood, little progress has been made in designing more effective therapies. For example, it has been proposed that the cytokine TNFα contributes to CHF pathogenesis, but the TNF-inhibitor Enbrel® has shown no efficacy in two trials in CHF, and is no longer being developed as a potential therapy.

Contraction of the heart is controlled through the cardiac action potential, the cyclic variation in cardiac muscle cell (cardiomyocyte) potential as sodium, potassium, and calcium ions diffuse through the cell membrane. During the cycle, the entry of calcium ions (Ca²⁺) into the cell triggers additional Ca²⁺ release from the sarcoplasmic reticulum (SR) into the cytosol. Cytoplasmic free (cytosolic) Ca²⁺ facilitates binding of thick and thin filaments of myofibrils inside the cardiomyocytes, leading to myofibrillar contraction and impulse generation. At the end of the cycle, Ca²⁺ is actively transported from the cytosol into the SR via the protein sarcoplasmic reticulum calcium ATPase (SERCA2a). Low cytosolic Ca²⁺ levels are required for myofibrillar relaxation. Persistently elevated cytosolic Ca²⁺ levels impair myofibrillar relaxation and may decrease force generation; it has therefore been proposed that Ca²⁺ overload in cardiac muscle cells plays a role in the pathogenesis of CHF.

Disordered Ca²⁺ signaling in cardiac myocytes may have other effects, including activation of Ca²⁺/calmodulin-dependent protein kinase II (M. E. Anderson et al., “Multifunctional Ca²⁺/calmodulin-dependent protein kinase mediates Ca²⁺-induced enhancement of the L-type Ca²⁺ current in rabbit ventricular myocytes,” Circulation Res. 75:854-861 (1994)). One consequence of calmodulin kinase II activation is augmentation of an inward Ca²⁺ current (L-current) that follows completion of action potential repolarization, further increasing Ca²⁺ overload and impairing cardiac muscle contractility. This is also an important predisposing condition for arrhythmia, a leading cause of cardiac-related deaths in North America and Europe. Arrhythmia following early after-depolarization has been shown, for example, in ischemic hearts (H. -C. Lee et al., “Effect of ischemia on calcium-dependent fluorescence transients in rabbit hearts containing indo 1,” Circulation 78:1047-1059 (1988)).

Activity of the SERCA2a protein is regulated by phospholamban, and in failing human hearts decreased phosphorylation of phospholamban has been reported (R. H. G. Schwinger et al., “Reduced Ca²⁺-sensitivity of SERCA2a in failing human myocardium due to reduced serin-16 phospholamban phosphorylation,” J. Mol. Cell. Cardiol. 31:479-491 (1999)). Deficient SERCA2a activity, which occurs during heart failure, results in abnormal Ca²⁺ handling and contractile dysfunction. It has been shown that increasing SERCA2a expression by gene transfer improves ventricular function in a rat model of heart failure, thereby improving cardiac metabolism and survival (F. del Monte et al., “Improvements in survival and cardiac metabolism after gene transfer of sarcoplasmic reticulum Ca²⁺-ATPase in a rat model of heart failure,” Circulation 104:1424-1429 (2001)). Additionally, overexpression of SERCA2a in human ventricular myocytes isolated from failing hearts corrects Ca²⁺ handling and improves contraction, as described in F. del Monte et al., “Restoration of contractile function in isolated cardiomyocytes from failing human hearts by gene transfer of SERCA2a,” Circulation 100:2308-2311 (1999).

Although these studies suggest that cardiac gene transfer is a promising treatment for CHF, a great deal more research is required before gene therapy becomes widely used in humans. It would be beneficial, therefore, to find additional therapies that address alternative mechanisms leading to Ca²⁺ overload and its role in CHF pathogenesis.

It has been proposed that immunologic and inflammatory responses contribute to the development of CHF. A strong positive correlation between levels of circulating chemokines and the severity of congestive heart failure has been described by P. Aukrust et al., “Elevated Circulating Levels of C-C Chemokines in Patients With Congestive Heart Failure,” Circulation 97:1136-1143 (1998). In particular, levels of monocyte chemoattractant protein-1 (MCP-1) are highly correlated with the severity of CHF and inversely correlated with left ventricular ejection fraction. The authors proposed that MCP-1 increases oxygen radical production by monocytes and thereby induces apoptosis of myocardial cells.

Experimental animal studies also support the interpretation that MCP-1 contributes to CHF pathogenesis. Transgenic over-expression of MCP-1 in the myocardium of mice resulted in myocarditis and the subsequent development of heart failure (P. E. Kolattukudy et al., “Myocarditis induced by targeted expression of the MCP1 gene in murine cardiac muscle,” Am. J. Pathol. 152:101-111 (1998)). In rats with pressure overload or volume overload, increased expression of MCP-1 in the myocardium accompanies the development of progressive cardiac decompensation and CHF, as described in T. Shioi et al., “Increased expression of interleukin-1β and monocyte chemotactic and activating factor/monocyte chemoattractant protein-i in the hypertrophied and failing heart with pressure overload,” Circ. Res. 81:664-671 (1997), and T. M. Behr et al., “Monocyte chemoattractant protein-1 is upregulated in rats with volume-overload congestive heart failure,” Circulation 102:1315-1322 (2000).

As yet, no therapies have been developed based on the role of chemokines in the pathogenesis of CHF.

### SUMMARY OF THE INVENTION

The present invention provides methods and compositions for treating congestive heart failure (CHF) and related conditions in mammals. In addition to their monocyte-recruiting function, CC chemokines may have a more direct role in CHF pathogenesis. In other cell types, the binding of CC chemokines to receptors on target cells activates a G-protein-coupled signal transduction system, leading to an increase in cytosolic Ca²⁺ concentrations. Binding of CC chemokines to receptors on cardiomyocytes may, in a similar manner, lead to Ca²⁺ overload. According to the present invention, a protein binding several CC chemokines with high affinity is used to prevent chemokine-mediated disruption of Ca²⁺ signaling and the resultant impairment of cardiac muscle contractility that leads to CHF.

Since several chemokines (e.g., MCP-1, MIP-1α, and RANTES) may have synergistic effects in the pathogenesis of CHF, blocking the effect of any one of these may be insufficient to treat the disorder. In methods of the invention, CHF in a mammal is treated by administering to the mammal a therapeutically effective amount of a protein binding more than one chemokine. Preferably, the protein binds the CC chemokines MCP-1, MIP-1α, and RANTES with high affinity. In one embodiment, the protein is a poxyirus-encoded chemokine inhibitor protein, which can be encoded by a virus such as cowpox, vaccinia, variola, myxoma, or Shope fibroma. In an alternative embodiment, the protein is cytomegalovirus receptor U28, human receptor D6, or any other protein binding several chemokines. According to the present invention, several CC chemokines may have synergistic effects with one another and with other mechanisms such as activation of Ca²⁺/calmodulin-dependent protein kinase II to produce Ca²⁺ overload. Elimination of the contribution by several CC chemokines may be sufficient to restore normal contractile function.

The present invention also provides a pharmaceutical composition containing an amount of a chemokine-binding protein sufficient to ameliorate the augmenting effect of CC chemokines on cytosolic Ca²⁺ concentrations in cardiac muscle cells. The protein binds several CC chemokines such as MCP-1, MIP-1α, and RANTES. In one embodiment, the protein is a poxyirus-encoded chemokine inhibitor, encoded by a virus such as cowpox, vaccinia, variola, myxoma, or Shope fibroma. Alternatively, the protein can be cytomegalovirus receptor U28, human receptor D6, or any other protein binding several CC chemokines.

Also provided by the present invention is a method for identifying a compound, e.g., a protein, that modulates the activity of at least one, and preferably more than one, CC chemokine, such as MCP-1, MIP-1α, and RANTES. In this method, isolated cardiomyocytes are exposed to at least one CC chemokine in the presence or absence of a compound suspected of modulating the activity of the CC chemokine or chemokines. Under conditions suitable for measuring L-type Ca²⁺ current, the cells are stimulated to increase the cytosolic Ca²⁺ concentration. The resulting Ca²⁺ transients in the presence and absence of the compound are compared to determine whether the compound modulates activity of the CC chemokine or chemokines. The present invention also includes compounds identified by this method.

### BRIEF DESCRIPTION OF THE FIGURE

FIG. 1 is a plot of Ca²⁺ transients in isolated rabbit ventricular muscle cells, assayed with the Ca²⁺ reporter Fluo-3, following exposure to a control solution and to the chemokine MCP-1 at varying concentrations and exposure times.

### DETAILED DESCRIPTION OF THE INVENTION

The present invention provides methods for treating congestive heart failure (CHF) and related conditions such as cardiac arrhythmia by administering a chemokine-binding protein to a mammal having the disease. The protein binds to multiple CC chemokines, preventing their interaction with receptors on cardiomyocytes. Because this interaction is believed to augment the concentration of cytosolic Ca²⁺, the method prevents or ameliorates the chemokine-induced disruption of Ca²⁺ signaling. Normal heart muscle contractility is thereby restored.

Chemokines (chemotactic cytokines) are a family of small molecular mass proteins (8 to 16 kD) that are classified into four subfamilies on the basis of their N-terminal amino acid sequence and the type of leukocyte they attract. The CXC or α chemokines, which attract and activate neutrophils, have a single amino acid inserted between the first and second of four cysteine residues. These cysteines are not separated in the CC or β chemokines, which are potent chemoattractants of monocytes. The two other subfamilies are C or γ chemokines, which have only a single pair of cysteines, and CX₃C chemokines, which have three amino acid residues separating the first and second cysteine residues. The mechanism of CC chemokine action involves initial binding to specific transmembrane G-protein-coupled receptors on target cells and activation of a signal transduction system leading to increased cytosolic Ca²⁺.

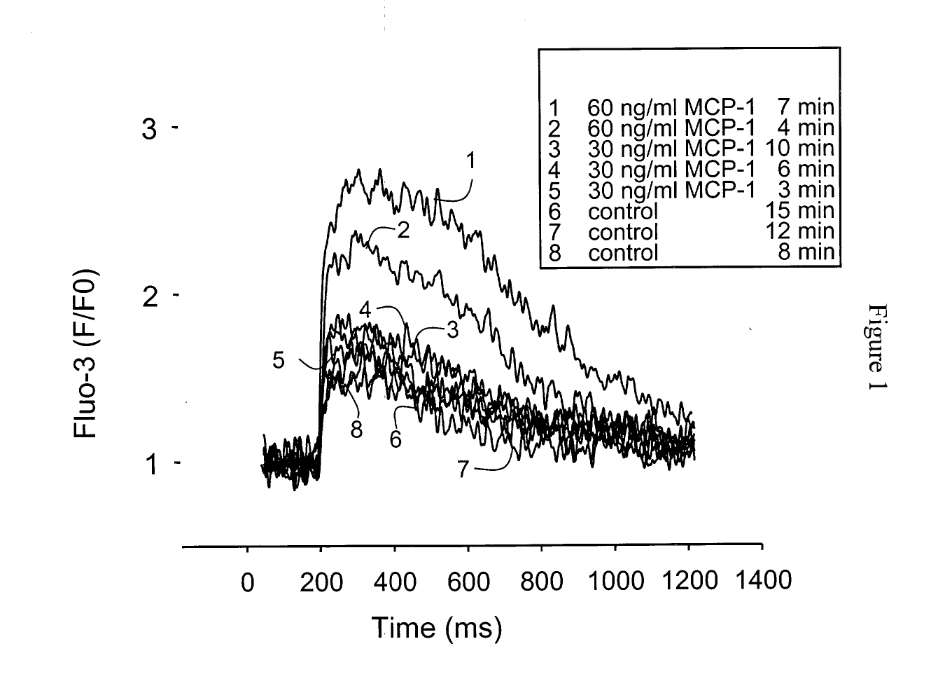

As determined by the present inventor, chemokines may contribute to Ca²⁺ overload in cardiac muscle cells and the resulting CHF. Binding of CC chemokines to receptors on cardiomyocytes involves a G-protein-coupled signal transduction system that may lead to an increase in cytosolic Ca²⁺. This, in turn, may lead to Ca²⁺ release from the sarcoplasmic reticulum (SR). The combined effect of L-channel opening and chemokines may result in cytosolic Ca²⁺ levels above those that can be restored by pumping Ca²⁺ back into the SR, particularly if the activity of SERCA2a is suboptimal. The resulting Ca²⁺ overload impairs contractile function of the cells. This mechanism has not been observed previously in cardiomyocytes, but occurs in other types of cells such as monocytes and sarcoma cells (B. -S. Youn et al., “Characterization of CKβ8 and CKβ8-1: Two Alternatively Spliced Forms of Human β-Chemokine, Chemoattractants for Neutrophils, Monocytes, and Lymphocytes, and Potent Agonists at CC Chemokine Receptor 1,” Blood 91:3118-3126 (1998)). It is likely (see FIG. 1) that the same process occurs in cardiomyocytes, and that chemokines have a more direct effect on the cells than previously proposed.

FIG. 1 is a plot of Ca²⁺ transients in isolated rabbit ventricular myocytes, assayed by the Ca²⁺-reporter dye Fluo-3. In this study, cultured myocytes were incubated with Fluo-3 using solution and voltage clamp conditions that allow measurement of the L-type Ca²⁺ current. Different concentrations of the CC chemokine monocyte chemoattractant protein 1 (MCP-1) or a control solution were added for varying times. Cells were then stimulated, inducing release of Ca²⁺ from the sarcoplasmic reticulum and inward Ca²⁺ flux. Changes in the amplitude of the signal in FIG. 1 reflect Ca²⁺ transients, i.e., the rise of intracellular Ca²⁺ concentration upon stimulation. As shown, MCP-1 increased the magnitude of the Ca²⁺ transients in a concentration- and time-dependent manner. The results indicate a strong correlation between the CC chemokine MCP-1 and the cytosolic Ca²⁺ concentration, indicating that a chemokine inhibitor may decrease the cytosolic Ca²⁺ concentration, thereby preventing Ca²⁺ overload in the cardiac muscle cells. Experimental methods used to obtain FIG. 1 are described (without chemokine addition) in M. E. Anderson et al., “Multifunctional Ca²⁺/calmodulin-dependent protein kinase mediates Ca²⁺-induced enhancement of the L-type Ca²⁺ current in rabbit ventricular myocytes.,” Circ. Res. 75:854-861 (1994), incorporated herein by reference.

Because the same chemokine receptor transduction pathway is activated by several CC chemokines, each binding to its receptor, it is likely that other CC chemokines are likewise mediators of Ca²⁺ overload in myocardial cells and of CHF. Thus it is improbable that an antagonist to the binding of a single chemokine to its receptor would be sufficient to prevent Ca²⁺ overload. An inhibitor of several CC chemokines, however, may prevent Ca²⁺ overload. CC chemokines include macrophage inflammatory proteins 1α and 1β (MIP-1α, MIP-1β), RANTES (regulated on activation, normal T-cell expressed and secreted protein), MCP-1, MCP-2, and MCP-3, among others.

In methods of the invention, a therapeutically effective amount of an isolated, promiscuous chemokine-binding protein is administered to a subject to treat congestive heart failure, arrhythmia, or related disorders of cardiac contraction. As used herein, a promiscuous chemokine-binding protein is one that binds with non-negligible affinity to more than one different chemokine. The protein can therefore inhibit the binding of several CC chemokines to cardiac myocytes and chemokine-induced functional responses in these cells, including cytosolic Ca²⁺ mobilization through G-protein-coupled mechanisms. As a result, the disruption of cytosolic Ca²⁺ levels caused by binding of chemokines is ameliorated or prevented.

In a preferred embodiment of the invention, the chemokine-binding protein is a poxyirus-encoded CC chemokine inhibitor (vCCI) protein. A number of virus-encoded proteins have been identified that bind to CC chemokines with high affinity, in some cases with higher affinity than that of the chemokines to their receptors.

Suitable poxyirus-encoded proteins are described in C. A. Smith et al., “Poxyirus Genomes Encode a Secreted, Soluble Protein That Preferentially Inhibits β Chemokine Activity yet Lacks Sequence Homology to Known Chemokine Receptors,” Virology 236:316-327 (1997), incorporated herein by reference. These homologous proteins bind to a wide range of CC chemokines, including MCP-1, MIP-1α, and RANTES, with affinities in the nanomolar range. However, they share no sequence homology with known chemokine receptors. A number of these proteins have been shown to inhibit the proinflammatory activity of CC chemokines completely. In a recent study mapping the binding capacity of a particular vCCI to chemokines (J. M. Bums et al., “Comprehensive Mapping of Poxyirus vCCI Chemokine Binding Protein: Expanded Range of Ligand Interactions and Unusual Dissociation Kinetics,” J. Biol. Chem. 277:2785-2789 (2002), incorporated herein by reference), the protein was shown to bind with high affinity to most CC chemokines but not to other chemokine subfamilies.

Most poxyiruses express a vCCI protein. One such protein is encoded by the cowpox virus. An additional suitable protein is encoded by an open reading frame in the genome of the Lister strain of vaccinia virus, described in A. H. Patel et al., “DNA sequence of the gene encoding a major secreted protein of vaccinia virus, strain Lister,” J. Gen. Virol. 71:2023-2031 (1990), incorporated herein by reference. Another suitable protein is encoded by an open reading frame in the genome of the variola (smallpox) virus. DNA and encoded amino acid sequences for these three poxyirus-encoded proteins, referred to as p35 proteins, are provided in U.S. Pat. No. 5,871,740, issued to Smith, incorporated herein by reference. All variations of the proteins described in the Smith patent can be used in methods of the present invention, i.e., all polypeptides that are substantially homologous to the p35 proteins listed therein and that exhibit the desired chemokine-binding property. Additionally, homologs derived from higher organisms, such as mammalian cells, can also be used.

Another suitable poxyirus-encoded chemokine inhibitor is the myxoma M-T7 protein, described in U.S. Pat. No. 5,834,419, issued to McFadden et al., incorporated herein by reference. This protein is encoded by the M-T7 open reading frame of the myxoma virus, and its sequence is provided in the McFadden patent. Proteins used in methods of the present invention include all variations described in the McFadden patent.

As will be apparent to those of skill in the art, the present invention includes methods employing any viral chemokine inhibitor protein or its homolog, including those not currently identified. For example, suitable proteins are encoded by the genomes of myxoma virus, cowpox, Shope fibroma virus, ectromelia, rabbitpox, and other mammalian poxyiruses. Additionally, any protein that is substantially homologous to those listed herein and has sufficient chemokine inhibiting activity can be employed.

In an alternative embodiment of the invention, the promiscuous chemokine-protein binding protein is the human D6 (hD6) chemokine receptor described in R. J. B. Nibbs et al., “Cloning and Characterization of a Novel Promiscuous Human β-Chemokine Receptor D6,” J. Biol. Chem. 272:32078-32083 (1997), incorporated herein by reference. hD6 is a highly promiscuous CC chemokine receptor that displays relatively high affinity binding for most CC chemokines. Methods of the present invention can use any polypeptide that is substantially homologous to the hD6 protein and that exhibits the desired chemokine-binding property. The amino acid sequence of the hD6 protein is provided in the Nibbs reference.

Alternatively, the protein can be the chemokine receptor US28, encoded by the US28 open reading frame of the human cytomegalovirus. This protein is a receptor for RANTES, MIP-1α, and MCP-1, but not for CXC chemokines, as described in J. L. Gao et al., “Human cytomegalovirus open reading frame US28 encodes a functional beta chemokine receptor,” J. Biol. Chem. 269:28539-28542 (1994), incorporated herein by reference.

In an alternative embodiment, the protein is the promiscuous chemokine receptor described in U.S. Pat. No. 6,150,132, issued to Wells et al., incorporated herein by reference. This receptor binds to MCP-1, MIP-1α, and RANTES, and its amino acid sequence is provided in the Wells patent. Proteins used in methods of the present invention include all variations described in the Wells patent.

In methods of the invention, a therapeutically effective amount of a promiscuous chemokine-binding protein (e.g., a poxyirus-encoded chemokine inhibitor protein or chemokine receptor protein) is administered to a mammal suffering from or at risk of congestive heart failure. Any of the above-described proteins can be employed, including native proteins; variants, derivatives, oligomers, and biologically active fragments thereof; and fusion proteins containing the chemokine-binding protein. The protein may act to diminish the increase of free Ca in the cytoplasm of myocardiocytes, thereby avoiding the chemokine-induced disruption of the myofibrillar contraction and relaxation cycle.

The present invention also provides methods for treating any condition related to impaired cardiomyocyte contractility caused by chemokine-mediated Ca²⁺ signaling disruption. One example is cardiac arrhythmia. In these methods, a therapeutically effective amount of a promiscuous chemokine-binding protein is administered.

The chemokine-binding protein is administered by any suitable means, preferably by intravenous injection, continuous infusion, inhalation, subcutaneous injection, or sustained release from implants (e.g., gels or membranes). The protein is typically administered in the form of a pharmaceutical composition, formulated according to known methods used to prepare pharmaceutically useful compositions. For example, the composition can include an effective amount of a purified chemokine-binding protein and a pharmaceutically acceptable diluent, excipient, or carrier. Such carriers are nontoxic to patients at the dosages and concentrations employed. The composition can also include suitable emulsifiers or preservatives. Typically, the composition is prepared by combining the protein with buffers, antioxidants such as ascorbic acid, low molecular weight peptides, proteins, amino acids, carbohydrates including glucose, sucrose, or dextran, chelating agents such as EDTA, glutathione, or other stabilizers and excipients.

The chemokine inhibitor protein can also be coupled with polyethylene glycol or incorporated into polymeric compounds or into liposomes, microemulsions, micelles, unilamellar or multilamellar vesicles, erythrocyte ghosts, or spheroblasts. Because these compositions influence the physical state, solubility, stability, rate of in vivo release, and rate of in vivo clearance of the protein, they are chosen accordingly.

The pharmaceutical compositions are administered in a manner and dosage appropriate to the age, sex, condition, and extent of disease of the patient. The amount of protein administered is a therapeutically effective amount, i.e., an amount that is effective in ameliorating the condition. Generally, an effective amount is an amount effective either (1) to reduce the symptoms of the disease sought to be treated or (2) to induce a pharmacological change relevant to treating the disease sought to be treated. For CHF, an effective amount includes an amount effective to: increase the heart's ability to contract; reduce fluid retention; reduce the concentration of cytosolic Ca²⁺ in cardiomyocytes; or increase the life expectancy of the affected mammal. As used herein, ameliorating refers to a lessening of the detrimental effect or the symptoms of congestive heart failure or related condition in the subject being treated. This amelioration typically occurs by reducing the increase of cytosolic Ca²⁺ in cardiac myocytes as triggered by CC chemokines. Thus in some cases, a therapeutically effective amount is an amount effective to prevent or lessen chemokine-mediated disturbances of Ca²⁺ signaling in cardiac myocytes in congestive heart failure or arrhythmias. This amount typically varies for patients based on age, sex, condition, extent of disease, and other factors. Suitable dosage ranges are 10-1000 mg, preferably 20-500 mg, and more preferably 50-200 mg.

Proteins used in methods and compositions of the invention can be produced by recombinant or non-recombinant methods. Non-recombinant proteins can be purified from the culture supernatant of cells infected with vCCI-encoding viruses. In recombinant methods, expression vectors encoding the promiscuous chemokine-binding protein are introduced into host cells. The vectors include the protein-encoding sequence operably linked to suitable transcriptional or translational regulatory nucleotide sequences, such as those derived from a mammalian, microbial, viral, or insect gene. Suitable host cells include prokaryotes, yeast, or higher eukaryotic cells. Chemokine-binding proteins can be produced using standard recombinant methods. The proteins are then purified before use. Suitable methods for producing poxyirus-encoded chemokine inhibitors are described in detail in U.S. Pat. Nos. 5,871,740 and 5,834,419, described above. Production methods for the chemokine receptor proteins are found in Nibbs et al., Gao et al., and U.S. Pat. No. 6,150,132, cited above.

Variants and derivatives of any vCCI or chemokine receptor proteins that retain the desired biological activity can be used in methods of the present invention. Variants can be obtained by mutation of nucleotide sequences coding for native polypeptides, for example. Variants are polypeptides that are substantially homologous to native proteins but have amino acid sequences that differ by one or more deletions, insertions, or substitutions.

Preferably, the promiscuous chemokine-binding protein is an isolated chemokine-binding protein. As used herein, an isolated or biologically pure protein is a protein that has been removed from its natural environment. As such, the terms “isolated” and “biologically pure” do not necessarily reflect the extent to which the protein has been purified. Isolated proteins of the present invention are preferably retrieved in substantially pure form. As used herein, “substantially pure” refers to a purity that allows for the effective use of the protein in a functional assay, e.g., a chemokine-binding assay. As used herein, an isolated chemokine-binding protein can be a full-length modified protein or any homolog of such a protein. It can also be (e.g., for a pegylated protein) a modified full-length protein or a modified homolog of such a protein.

Proteins used in methods of the present invention preferably contain amino acid sequences that are at least about 75%, more preferably at least about 80%, more preferably at least about 85%, more preferably at least about 90%, more preferably at least about 95%, and most preferably at least about 98% identical to amino acid sequences provided in the references listed above, or to a protein encoded by an allelic variant of a nucleic acid molecule encoding a protein containing any of these sequences. Methods to determine percent identities between amino acid sequences and between nucleic acid sequences are known to those skilled in the art. Preferred methods to determine percent identities between sequences include computer programs such as the GCG® Wisconsin package™ (available from Accelrys Corporation), the DNAsis™ program (available from Hitachi Software, San Bruno, Calif.), the Vector NTI Suite (available from Informax, Inc., North Bethesda, Md.), or the BLAST software available on the NCBI website.

Promiscuous chemokine-binding proteins of the present invention may be identified by their ability to perform the function of a chemokine-binding protein subunit in a functional assay, e.g., in a chemokine-binding assay or a Ca²⁺ flux assay as described above. The phrase “capable of performing the function of that protein in a functional assay” means that the protein has at least about 10% of the activity of the natural protein in the functional assay, more preferably at least about 20% of the activity, more preferably at least about 30% of the activity, more preferably at least about 40% of the activity, more preferably at least about 50% of the activity, more preferably at least about 60% of the activity, more preferably at least about 70% of the activity, more preferably at least about 80% of the activity, and most preferably at least about 90% of the activity.

In one embodiment of the invention, the administered protein is a fusion protein of the chemokine-binding protein and an Fc region polypeptide derived from an antibody. The proteins are fused in a manner that does not substantially affect the binding of chemokines to the chemokine-binding protein. A similar fusion protein of a TNF receptor and Fc has been used successfully for treating rheumatoid arthritis. Fusion proteins can be produced using standard methods, e.g., by creating an expression vector encoding the chemokine-binding protein fused to the antibody polypeptide and inserting the vector into a suitable host cell. Suitable Fe polypeptides include the native Fe region polypeptide derived from a human IgG1 or the Fe mutein described in U.S. Pat. No. 5,457,035, issued to Baum et al. These methods are further described in U.S. Pat. No. 5,871,740.

In an alternative embodiment, the chemokine-binding protein can be substituted for the variable portion of an antibody heavy or light chain. If fusion proteins are made with both heavy and light chains of an antibody, it is possible to form an oligomer with up to four polypeptides. An oligomeric protein can also be made by joining two or more polypeptides through peptide linkers using conventional recombinant DNA technology.

Chemokine-binding fragments of the proteins, rather than the full proteins, can also be employed in methods of the invention. Fragments may be less immunogenic than the corresponding full-length proteins. The ability of a fragment to bind chemokines can be determined using a standard assay. Fragments can be prepared by any of a number of conventional methods. For example, a desired DNA sequence can be synthesized chemically or produced by restriction endonuclease digestion of a full length cloned DNA sequence and isolated by electrophoresis on agarose gels. Linkers containing restriction endonuclease cleavage sites can be employed to insert the desired DNA fragment into an expression vector, or the fragment can be digested at naturally-present cleavage sites. The polymerase chain reaction (PCR) can also be employed to isolate a DNA sequence encoding a desired protein fragment. Oligonucleotides that define the termini of the desired fragment are used as 5′ and 3′ primers in the PCR procedure. Additionally, known mutagenesis techniques can be used to insert a stop codon at a desired point, e.g., immediately downstream of the codon for the last amino acid of the desired fragment.

In an alternative embodiment, the protein is coupled to polyethylene glycol (PEG) to decrease loss into the urine and proteolytic degradation, thereby augmenting the duration of efficacy, and to reduce immunogenicity. The pegylated compound is administered to a patient as a therapy for congestive heart failure. Such modification may be desirable if the protein is to be administered repeatedly to an individual. Pegylation has been shown to reduce the immunogenicity of a number of proteins as well as to increase the serum half-life and solubility of certain proteins. PEG can be covalently linked to lysine or cysteine residues, to carbohydrate moieties on glycosylated proteins, or selectively to the N-terminus of proteins. Pegylation is described in F. M. Veronese, “Peptide and protein PEGylation: a review of problems and solutions,” Biomaterials 22:405-417 (2001), incorporated herein by reference.

The present invention also provides a method of screening for or identifying a compound that modulates the activity of at least one, and preferably more than one, CC chemokine. In this method, the same assay is performed as described above with reference to FIG. 1. In this case, however, the chemokine or chemokines are added along with a compound suspected of modulating the chemokine activity. Ca²⁺ transients are measured as described above and compared in the presence and absence of the compound, both with and without added chemokine. A decrease in the Ca²⁺ transient in the presence of the compound and chemokine, with respect to the level in the presence of chemokine only, indicates that the compound modulates the activity of the chemokine. Also included within the scope of the present invention are compounds, such as proteins, that modulate the activity of CC chemokines as identified by this method.

It should be noted that the foregoing description is only illustrative of the invention. Various alternatives and modifications can be devised by those skilled in the art without departing from the invention. Accordingly, the present invention is intended to embrace all such alternatives, modifications and variances which fall within the scope of the disclosed invention.

What is claimed is:

1. A method for treating congestive heart failure in a mammal, comprising administering to said mammal a therapeutically effective amount of a chemokine-binding protein.

2. The method of claim 1, wherein said chemokine-binding protein binds more than one CC chemokine.

3. The method of claim 2, wherein said CC chemokines are selected from the group consisting of MCP-1, MIP-1α, and RANTES.

4. The method of claim 1, wherein said chemokine-binding protein is a poxvirus-encoded chemokine inhibitor protein.

5. The method of claim 4, wherein said poxyirus-encoded chemokine inhibitor protein is encoded by a virus selected from the group consisting of cowpox, vaccinia, variola, myxoma, or Shope fibroma.

6. The method of claim 1, wherein said chemokine-binding protein is a chemokine receptor protein.

7. The method of claim 6, wherein said chemokine receptor protein is human receptor D6.

8. The method of claim 6, wherein said chemokine receptor protein is cytomegalovirus receptor U28.

9. A pharmaceutical composition comprising an amount of an isolated chemokine-binding protein effective to ameliorate the effect of CC chemokines on cytosolic Ca²⁺ concentration in cardiac muscle cells.

10. The composition of claim 9, wherein said chemokine-binding protein binds more than one CC chemokine.

11. The composition of claim 10, wherein said CC chemokines are selected from the group consisting of MCP-1, MIP-1α, and RANTES.

12. The composition of claim 9, wherein said chemokine-binding protein is a poxyirus-encoded chemokine inhibitor.

13. The composition of claim 12, wherein said poxyirus-encoded chemokine inhibitor is encoded by a virus selected from the group consisting of cowpox, vaccinia, variola, myxoma, or Shope fibroma.

14. The composition of claim 9, wherein said chemokine-binding protein is a chemokine receptor protein.

15. The composition of claim 14, wherein said chemokine receptor protein is human receptor D6.

16. The composition of claim 14, wherein said chemokine receptor protein is cytomegalovirus receptor U28.

17. A method for identifying a compound that modulates the activity of at least one CC chemokine on cardiomyocytes, comprising:
a) contacting a compound suspected of modulating the activity of at least one CC chemokine with cardiac myocytes in the presence of said at least one CC chemokine and under conditions suitable for measuring L-type Ca²⁺ transients;
b) stimulating said myocytes to increase cytosolic Ca²⁺ concentrations;
c) repeating steps (a) and (b) in the absence of said compound;
d) comparing cytosolic Ca²⁺ concentrations in the presence of said compound with cytosolic Ca²⁺ concentrations in the absence of said compound; and
e) based on said comparison, determining whether said compound modulates the activity of said at least one CC chemokine.

18. The method of claim 17, wherein said compound is a protein.

19. The method of claim 17, wherein said compound binds more than one CC chemokine.

20. The method of claim 17, wherein said at least one CC chemokine is selected from the group consisting of MCP-1, MIP-1α, and RANTES.

21. A compound identified by the method of claim 17.
